(19) United States
(12) Patent Application Publication
(10) Pub. No.: US 2006/0217356 A1
(43) Pub. Date: Sep. 28, 2006

(54) Nutritional supplement for lowering serum triglyceride and cholesterol levels

(76) Inventors: Jeffrey L.C. Wright, Wilmington, NC (US); Jaroslav A. Kralovec, Halifax (CA)

Correspondence Address: NEEDLE & ROSENBERG, P.C., SUITE 1000, 999 PEACHTREE STREET, ATLANTA, GA, 30309-3915, US

(21) Appl. No.: 11/371,113

(22) Filed: Mar. 8, 2006

Related U.S. Application Data
(63) Continuation of application No. 09/385,834, filed on Aug. 30, 1999, now abandoned.

(51) Int. Cl. A61K 31/56; A61K 31/202

(52) U.S. Cl. 514/171; 514/560

(57) ABSTRACT

Triglycerides and cholesterol in the bloodstream are important factors in the development in the development of cardiovascular disease. The present invention discloses a nutritional supplement comprising a sterol and an omega-3 fatty acid, or an ester thereof, for lowering cholesterol and triglyceride levels in the bloodstream of a subject. Preferably, the sterol and omega-3 fatty acid are together in the form of an ester.

### FIELD OF THE INVENTION

The invention relates to control of cholesterol and triglyceride levels in mammals, particularly humans.

### BACKGROUND OF THE INVENTION

Serum cholesterol and serum triglyceride levels are important factors in the development of cardiovascular disease. In many clinical studies there is a positive correlation between plasma triglycerides and the incidence of cardiovascular disease [1]. Elevated plasma triglyceride level is frequently associated with other atherogenic factors including elevated low-density lipoprotein (LDL)-cholesterol, reduced high-density lipoprotein (HDL)-cholesterol, and small LDL particles [2, 3]. There is growing acceptance that triglycerides act in a synergistic fashion with these other lipid risk factors to increase the incidence of cardiovascular disease [4, 5]. Hypertriglyceridemia usually occurs because of insulin resistance, which leads to overproduction of very low-density lipoproteins (VLDL) by the liver [3]. Treatment involves lifestyle changes to decrease body weight and to increase physical activity, both of which improve insulin sensitivity. Drug therapy to lower triglycerides involves the use of fibrates or nicotinic acid [6].

A number of clinical studies convincingly establish plasma cholesterol and LDL-cholesterol as independent risk factors for coronary heart disease [7]. Pharmacological agents, called statins, lower total plasma cholesterol by inhibiting the synthesis of cholesterol by the liver. The statins reduce the morbidity and mortality rate from cardiovascular disease in high risk, hypercholesterolemic patients [8, 9], but also in persons who exhibit “average” cholesterol levels [10]. Another approach is to interfere with the intestinal absorption of cholesterol. Certain phytosterols (plant sterols) such as stigmasterol and β-sitosterol lower serum cholesterol act by inhibiting absorption of both dietary and biliary cholesterol from the small intestine [11].

With respect to the most appropriate form of phytosterols for lowering serum cholesterol, some reports indicate that free phytosterols reduce serum cholesterol in animals and humans [12, 13]. However, there is also evidence to indicate that a sterol esterified with a fatty acid may be more effective [14]. Trials show that phytosterol esters of plant fatty acids obtained from canola oil, when incorporated into food such as margarine or mayonnaise, lower total cholesterol and LDL-cholesterol levels by about 10 and 15 percent, respectively [15, 16]. U.S. Pat. No. 5,502,045 (Miettinen et al., issued Mar. 26, 1996) discloses the use of sitostanol esters of canola oil to lower serum cholesterol. Benecol™ (Raisio Benecol Ltd., Raisio, Finland), a margarine that contains such compounds, is now on the market.

The mechanism by which phytosterols or phytosterol esters inhibit absorption of dietary cholesterol by the digestive tract is not fully understood but may involve competitive inhibition of cholesterol uptake from the Intestinal lumen or inhibition of cholesterol esterification in the intestinal mucosa [12]. It is known that phytosterols themselves are only poorly absorbed. Vanhanen et al. [17] report that phytosterol esters may also be poorly absorbed by the intestinal tract based on postprandial measurements of β-sitostanol in plasma. A direct measure of phytosterol ester uptake by the digestive tract has not been reported.

When phytosterols are esterified with fatty acids from plant sources such as canola, the long-chain polyunsaturated fatty acids (LCPUFAs) that are incorporated are predominantly of the omega-6 series. Omega-6 fatty acids do not affect plasma triglycerides. Research to date on fatty acid esters of sterols has focused only on the efficacy of the sterol in lowering cholesterol.

### SUMMARY OF THE INVENTION

The present invention provides a nutritional supplement comprising a sterol and an omega-3 fatty acid, or an ester thereof, for lowering cholesterol and triglyceride levels in the bloodstream of a subject.

The present invention also provides a method of lowering cholesterol and triglyceride levels in the bloodstream of a subject, the method including the step of administration of an effective amount of a nutritional supplement comprising a sterol and an omega-3 fatty acid, or an ester thereof, to a subject.

The present invention also provides the use of the nutritional supplement defined herein for lowering cholesterol and triglyceride levels in the bloodstream of a subject.

The present invention further provides a foodstuff composition comprising the nutritional supplement defined herein and a foodstuff, the nutritional value of the foodstuff being enhanced by incorporation of the nutritional supplement defined herein.

The present invention further provides the use of the nutritional supplement defined herein in the manufacture of a foodstuff composition.

The subject is preferably a mammal, more preferably a human.

Sterols are not very soluble in lipid, which complicates their use in lipid-based foods. A mixture of a sterol and a free omega-3 fatty acid, which typically forms a paste at a molar ratio of 1:1, may be used. If a mixture is used, the omega-3 fatty acid can be a free acid or can be in ester form, preferably a succinimidyl, triglyceride, (C₃-C₁₂)cycloalkyl or (C₁-C₈)alkyl ester, more preferably an ethyl ester. In the mixture, the molar ratio range of omega-3 fatty acid, or an ester thereof, to sterol should be about 0.5 to 8 preferably 0.76 to 6.4, more preferably 1 to 2.

Preferably, the sterol and the omega-3 fatty acid are together in the form of an ester. The sterol esters of the present invention are highly fat-soluble and represent a bifunctional species, since they lower both serum cholesterol and serum triglyceride levels in the bloodstream.

### DETAILED DESCRIPTION OF THE PREFERRED EMBODIMENTS

The sterols used to prepare the nutritional supplement of the present invention are preferably phytosterols, and preferably have a perhydrocyclopentanophenanthrene ring system as shown below in the compound of formula I: 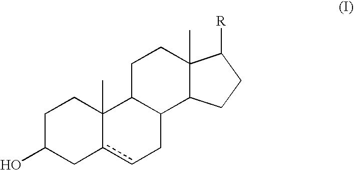
wherein the dashed line is a single or double bond and R is a (C₁-C₁₀)alkyl, substituted (C₁-C₁₀)alkyl, (C₂-C₁₀)alkenyl or substituted (C₂-C₁₀)alkenyl group.

In the present application, the term “sterols” includes sterols in reduced form (stanols), preferably β-sitostanol or fucostanol (reduced fucosterol).

One or more sterols can be used to prepare the nutritional supplement. The term “phytosterols” includes sterols from terrestrial or marine plants, seaweed, microalgae, etc. Preferably, the sterol is stigmasterol, sitosterol or fucosterol, as shown below, or β-sitostanol or fucostanol

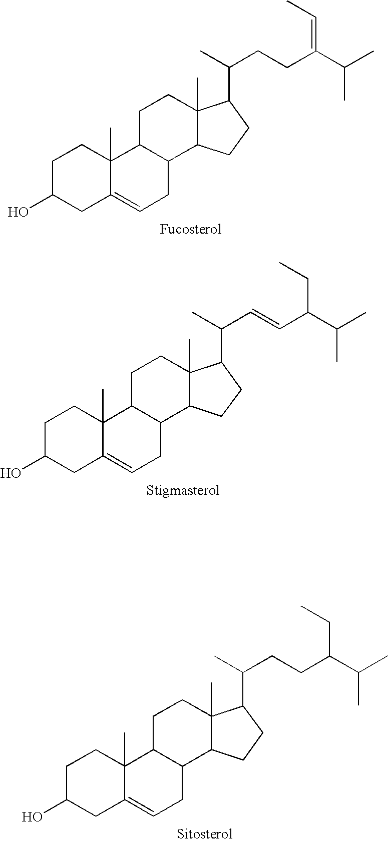

Fucosterol is abundant in brown algae. Prior to esterification with the omega-3 fatty acid, fucosterol can be reduced to fucostanol. Preferably, the reduction is carried out using hydrogen gas in the presence of a suitable catalyst such as palladium on charcoal (Pd/C), but other reduction processes that ultimately yield a food-quality ester, after purification if necessary, may be used.

The nutritional supplement of the present invention comprises one or more omega-3 fatty acids, and is preferably an ester of an acid of the formula: 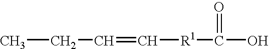
wherein R¹ is a (C₃-C₄₀)alkenyl group comprising at least one double bond, more preferably 2 to 5 double bonds. More preferably, the omega-3 fatty acid is stearidonic acid 18:4ω3 (SA), eicosapentaenoic acid 20:5ω3 (EPA) or docosahexaenoic acid 22:6ω3 (DHA).

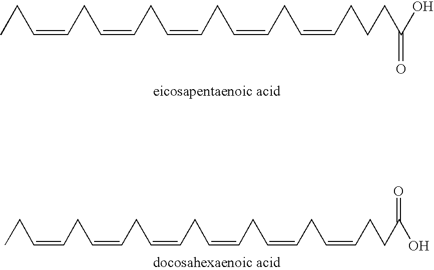

Omega-3 fatty acids, such as EPA and DHA, are long-chain polyunsaturated fatty acids (LCPUFAs) that are abundant in oily fish such as menhaden, salmon, tuna, and sardine, as well as in certain plants and microbes, such as articular fungi and microalgae. The preferred source of omega-3 fatty acids for the present invention is fish oil, more preferably a highly refined fish oil concentrate having approximately 65% omega-3 fatty acid content which is predominantly EPA and DHA in the form of triglyceride esters. These triglycerides are preferably converted to lower alkyl esters by known methods and used in an esterification with a sterol to form esters, which can be further purified if necessary, for use as nutritional supplements.

The cardiovascular effects of dietary fish oils have long been recognized [18, 19]. Omega-3 fatty acids lower plasma triglyceride concentrations principally by inhibiting synthesis of triacylglycerol and VLDL by the liver [20]. In addition, omega-3 fatty acids are anti-thrombotic and are protective against cardiac arrhythmias [21]. The benefits of fish oil consumption are illustrated by the finding of the Diet and Reinfarction Trial (DART) which showed a reduction of 29% in the overall mortality in survivors of a first myocardial infarction who consumed fish rich in omega-3 fatty acids at least twice weekly [22]. Two recent studies demonstrate the efficacy of omega-3 fatty acid supplementation. In a randomized, double-blind, placebo-controlled trial patients with coronary artery disease who ingested a 1.5 g/day fish oil supplement (55% EPA and DHA) for two years had less progression and more regression of their disease based on coronary angiography compared to patients ingesting the placebo [23]. In the GISSI-Prevenzione trial, omega-3 fatty acid supplements in patients who had myocardial infarction reduced cardiovascular death by 30% [24]. Although omega-3 fatty acids are anti-atherogenic, they do not lower plasma cholesterol and in some incidences may slightly increase LDL-cholesterol [25]. Safety and toxicological studies spanning several years have shown that fish oils are safe to consume. Recently, fatty acids such as the omega-3 fatty acids from fish oil were granted GRAS (Generally Regarded As Safe) status in the United States, which permits their addition to foods low in long-chain polyunsaturated fatty acids. The typical North American diet contains about 0.15 grams omega-3 fatty acids whereas Inuit may ingest up to 10 grams of omega-3 fatty acids daily. A daily intake of 2 to 3 grams of omega-3 fatty acids has consistently been shown to lower plasma triglycerides [18]. Therefore, a suitable daily intake of omega-3 fatty acid in the present invention is about 0.1 to about 10 grams, preferably about 2 to about 3 grams, but clearly greater amounts can be tolerated, and may be beneficial.

Phytosterols are considered safe for human consumption. A typical daily intake in North America is about 100 to 300 milligrams. However, a dose of greater than 3 grams of the phytosterol esters are required to have significant impact on plasma cholesterol levels [13]. Such doses are safe with no known side effects. In the present invention, a preferred daily intake of phytosterol is about 2 to about 3 grams.

Phytosterol esters prepared using fish oil as the source of omega-3 fatty acids contain a significant amount of EPA and DHA. Such esters can simultaneously reduce serum cholesterol and serum triglyceride levels. The triglyceride-lowering ability of the omega-3 fatty acid component of the ester is dependent on its entry into the circulatory system. A lipid esterase in the intestinal lumen may be responsible for release of the omega-3 fatty acid from the phytosterol, which would make both species available for uptake into the circulatory system. There is a non-specific lipid esterase, secreted into the intestinal lumen during digestion that is active against a variety of molecular species including cholesterol esters, monoglycerides, and esters of vitamin A [26].

At least one additive, such as listed below, can be included for consumption with the nutritional supplement of the invention and may have, for example, antioxidant, dispersant, antimicrobial, or solubilizing properties. A suitable antioxidant is, for example, vitamin C, vitamin E or rosemary extract. A suitable dispersant is, for example, lecithin, an alkyl polyglycoside, polysorbate 80 or sodium lauryl sulfate. A suitable antimicrobial is, for example, sodium sulfite or sodium benzoate. A suitable solubilizing agent is, for example, a vegetable oil such as sunflower oil, coconut oil, and the like, or mono-, di- or tri-glycerides.

Additives include vitamins such as vitamin A (retinol, retinyl palmitate or retinol acetate), vitamin B1 (thiamin, thiamin hydrochloride or thiamin mononitrate), vitamin B2 (riboflavin), vitamin B3 (niacin, nicotinic acid or niacinamide), vitamin B5 (pantothenic acid, calcium pantothenate, d-panthenol or d-calcium pantothenate), vitamin B6 (pyridoxine, pyridoxal, pyridoxamine or pyridoxine hydrochloride), vitamin B12 (cobalamin or cyanocobalamin), folic acid, folate, folacin, vitamin H (biotin), vitamin C (ascorbic acid, sodium ascorbate, calcium ascorbate or ascorbyl palmitate), vitamin D (cholecalciferol, calciferol or ergocalciferol), vitamin E (d-alpha-tocopherol, d-beta-tocopherol, d-gamma-tocopherol, d-delta-tocopherol or d-alpha-tocopheryl acetate) and vitamin K (phylloquinone or phytonadione).

Other additives include minerals such as boron (sodium tetraborate decahydrate), calcium (calcium carbonate, calcium caseinate, calcium citrate, calcium gluconate, calcium lactate, calcium phosphate, dibasic calcium phosphate or tribasic calcium phosphate), chromium (GTF chromium from yeast, chromium acetate, chromium chloride, chromium trichloride and chromium picolinate) copper (copper gluconate or copper sulfate), fluorine (fluoride and calcium fluoride), iodine (potassium iodide), iron (ferrous fumarate, ferrous gluconate or ferrous sulfate), magnesium (magnesium carbonate, magnesium gluconate, magnesium hydroxide or magnesium oxide), manganese (manganese gluconate and manganese sulfate), molybdenum (sodium molybdate), phosphorus (dibasic calcium phosphate, sodium phosphate), potassium (potassium aspartate, potassium citrate, potassium chloride or potassium gluconate), selenium (sodium selenite or selenium from yeast), silicon (sodium metasilicate), sodium (sodium chloride), strontium, vanadium (vanadium sulfate) and zinc (zinc acetate, zinc citrate, zinc gluconate or zinc sulfate).

Other additives include amino acids, peptides, and related molecules such as alanine, arginine, asparagine, aspartic acid, carnitine, citrulline, cysteine, cystine, dimethylglycine, gamma-aminobutyric acid, glutamic acid, glutamine, glutathione, glycine, histidine, isoleucine, leucine, lysine, methionine, ornithine, phenylalanine, proline, serine, taurine, threonine, tryptophan, tyrosine and valine.

Other additives include animal extracts such as cod liver oil, marine lipids, shark cartilage, oyster shell, bee pollen and d-glucosamine sulfate.

Other additives include unsaturated free fatty acids such as γ-linoleic, arachidonic and α-linolenic acid, which may be in an ester (e.g. ethyl ester or triglyceride) form.

Other additives include herbs and plant extracts such as kelp, pectin, Spirulina, fiber, lecithin, wheat germ oil, safflower seed oil, flax seed, evening primrose, borage oil, blackcurrant, pumpkin seed oil, grape extract, grape seed extract, bark extract, pine bark extract, French maritime pine bark extract, muira puama extract, fennel seed extract, dong quai extract, chaste tree berry extract, alfalfa, saw palmetto berry extract, green tea extracts, angelica, catnip, cayenne, comfrey, garlic, ginger, ginseng, goldenseal, juniper berries, licorice, olive oil, parsley, peppermint, rosemary extract, valerian, white willow, yellow dock and yerba mate.

Other additives include enzymes such as amylase, protease, lipase and papain as well as miscellaneous substances such as menaquinone, choline (choline bitartrate), inositol, carotenoids (beta-carotene, alpha-carotene, zeaxanthin, cryptoxanthin or lutein), para-aminobenzoic acid, betaine HCl, free omega-3 fatty acids and their esters, thiotic acid (alpha-lipoic acid), 1,2-dithiolane-3-pentanoic acid, 1,2-dithiolane-3-valeric acid, alkyl polyglycosides, polysorbate 80, sodium lauryl sulfate, flavanoids, flavanones, flavones, flavonols, isoflavones, proanthocyanidins, oligomeric proanthocyanidins, vitamin A aldehyde, a mixture of the components of vitamin A₂, the D Vitamins (D₁, D₂, D₃ and D₄) which can be treated as a mixture, ascorbyl palmitate and vitamin K₂.

The nutritional supplement of the invention is typically a viscous oil and can be added to a foodstuff composition during processing of the foodstuff. Such a foodstuff composition is often referred to as a functional food, and can be any food that will tolerate the physicochemical properties of the nutritional supplement, for example, margarine, cooking oil, shortening or mayonnaise. It can also be packaged for consumption in softgel, capsule, tablet or liquid form. It can be supplied in edible polysaccharide gums, for example carrageenan, locust bean gum, guar, tragacanth, cellulose and carboxymethylcellulose.

The nutritional supplement can also be microencapsulated. Microencapsulation can be carried out, for example, using a gelatin such as bovine gelatin in a co-extrusion process, prior to processing into a foodstuff composition, for example baked goods, candy, margarines and spreads, ice cream, yogurts, frozen desserts, cake mixes and pudding mixes. The packaging of the nutritional supplement should preferably provide physical protection from such effects as pH, particularly basic conditions, oxidation and degradation by light. This latter effect can be minimized for example by changing the mesh size of the microencapsulation or inclusion of a suitable dye. The nutritional supplement can also be stored in a light-opaque container to minimize photodegradation.

The example below describes synthesis of an ester of the invention. Esterification can be performed according to known methods, such as acid catalysis (U.S. Pat. No. 5,892,068: Higgins III, issued Apr. 6, 1999). Preferably however, a base is used to promote esterification, more preferably transesterification. More preferably, the base is a metal (C₁-C₁₀)alkoxide, even more preferably sodium methoxide or ethoxide.

### EXAMPLES

Synthesis of Stigmasterol/Omega-3 Fatty Acid Esters

A mixture of dry stigmasterol (3 g, 7.27 mmol) and a highly concentrated mixture of EPA and DHA omega-3 fatty acids in ethyl ester form (EPAX™ 5500, ProNova; 4.3 g, 12.6 mmol) were heated while being stirred magnetically at 140 to 145° C. for 2 hours under vacuum (5 mm). Subsequently the vacuum was disconnected and powdered sodium methoxide (40 mg, 0.75 mmol) was added quickly in one portion. The vacuum was connected immediately and the mixture was stirred at 140 to 145° C. for an additional 4 hours. Hexane (25 mL) was added to precipitate the residual stigmasterol and the mixture was centrifuged for 5 minutes at 15,000 g (0° C.), the supernatant was removed and the pellet was washed again with 5 mL of hexane. The remaining precipitate was centrifuged off and the supernatants combined. The organic phase was washed with water (5 mL), dried over sodium sulfate and the solvent removed under reduced pressure. TLC (hexane/diethylether/acetic acid (90:10:1), R_{f} 0.71. The yield was 5.9 g (85%). The ester product was a viscous oil.

When the experiment was repeated using freshly made sodium ethoxide, almost the same level of conversion was obtained as with sodium methoxide. However, this was not seen with commercially available sodium ethoxide, which performed more poorly than sodium methoxide.

### REFERENCES

1 Criqui, M. H. Triglycerides and cardiovascular disease: a focus on clinical trials. (1998) Eur Heart Journal 19 (Suppl A), A36-A39.
2 Grundy, S. M. Small LDL, atherogenic dyslipidemia, and the metabolic syndrome. (1997) Circulation 95, 1-4.
3 Grundy, S. M. Hypertriglyceridemia, atherogenic dyslipidemia, and the Metabolic Syndrome. (1998) Am J Cardiol 81, 18B-25B.
4 Gotto Jr., A. M. Triglyceride: the forgotten risk factor. (1998) Circulation 97, 1027-1028.
5 Jeppeson, J., Hein, O. H., Suadicani, P. and Gyntelberg, F. Triglyceride concentration and ischemic heart disease: an eight-year follow-up in the Copenhagen male study. (1998) Circulation 97, 1029-1036.
6 Franceschini, G. and Paoletti, R. Pharmacological control of hypertriglyceridemia. (1993) Cardiovasc Drugs Ther 7, 297-302.
7 Eisenberg, D. The importance of lowering cholesterol in patients with coronary heart disease. (1998) Clin Cardiol 21, 81-84.
8 Scandinavian Simvastatin Survival Study Group. Randomised trial of cholesterol lowering in 4444 patients with coronary heart disease: the Scandinavian Simvastatin Survival Study (4S). (1994) Lancet 344, 1383-1389.
9 Shepherd, J., Cobbe, S. M., Ford, I., Isles, C. G., Lorimer, A. R., MacFarlane, P. W., McKillop, J. H. and Packard, C. J. Prevention of coronary heart disease with pravastatin in men with hypercholesterolemia. (1995) N Engl J Med 333, 1301-1307.
10 Sacks, F. M., Pfeffer, M. A., Moye, L. A., Rouleau, J. L., Rutherford, J. D., Cole, T. G., Brown, L., Warnica, J. W., Arnold, J. M. O., Wun, C., Davis, B. R. and Braunwald, E. The effect of pravastatin on coronary events after myocardial infarction in patients with average cholesterol levels. (1996) N Engl J Med 335, 1001-1009.
11 Heinemann, T., Kullak-Ublick, G. A., Pietruck, B. and von Bergmann, K. Mechanisms of action of plant sterols on inhibition of cholesterol absorption: comparison of sitosterol and sitostanol. (1991) Eur J Clin Pharmacol 40 (Suppl 1), S59-S63.
12 Ling, W. H. and Jones, P. J. H. Dietary phytosterols: a review of metabolism, benefits and side effects. (1995) Life Sci 57, 195-206.
13 Jones, P. J. H., MacDougall, D. E., Ntanios, F. and Vanstone, C. A. Dietary phytosterols as cholesterol-lowering agents in humans. (1997) Can J Physiol Pharmacol 75, 217-227.
14 Vanhanen, H. T., Blomqvist, S., Ehnholm, C., Hyvonen, M., Jauhiainen, M., Torstila, I. and Miettnen, T. A. Serum cholesterol, cholesterol precursors, and plant sterols in hypercholesterolemic subjects with different apoE phenotypes during dietary sitostanol ester treatment. (1993) J Lipid Res, 1535-1544.
15 Heinemann, T., Leiss, O. and von Bergmann, K. Effect of low-dose sitostanol on serum cholesterol in patients with hypercholesterolemia. (1986) Atherosclerosis 61, 219-223.
16 Miettinen, T. A. and Gylling, H. Regulation of cholesterol metabolism by dietary plant sterols. (1999) Curr Opin Lipidol 10, 9-14.
17 Vanhanen, H. T., Kajander, J., Lehtovirta, H. and Miettinen, T. A. Serum levels, absorption efficiency, faecal elimination and synthesis of cholesterol during increasing doses of dietary sitostanol esters in hypercholesterolaemic subjects. (1994) Clin Sci 1994 87, 61-67.
18 Leaf, A. and Weber, P. C. Cardiovascular effects of n-3 fatty acids. (1988) N Engl J Med 318, 549-557.
19 Mishkel, G. J. and Cairns, J. A. Cardiovascular effects of w-3 polyunsaturated fatty acids (fish oils). (1990) Bailliere's Clin Haematol 3, 625-649.
20 Kinsella, J. E., Lokesh, B. and Stone, R. A. Dietary n-3 polyunsaturated fatty acids and amelioration of cardiovascular disease: possible mechanisms. (1990) Am J Clin Nutr 52, 1-28.
21 Connor, S. L. and Connor, W. E. Are fish oils beneficial in the prevention and treatment of coronary artery disease? (1997) Am J Clin Nutr 66 (Suppl), 1020S-1031S.
22 Burr, M. L., Fehily, A. M., Gilbert, J. F., Rogers, S., Holliday, R. M., Sweetnam, P. M., Elwood, P. C. and Deadman, N. M. Effects of changes in fat, fish, and fibre intakes on death and myocardial reinfarction: diet and reinfarction trial. (1989) Lancet 30, 757-761.
23 von Schacky, C., Angerer, P., Kothny, W., Theisen, K. and Mudra, H. The effect of dietary omega-3 fatty fcids on coronary atherosclerosis: A randomized, double-blind, placebo-controlled trial. (1999) Ann Intern Med 130, 554-562.
24 GISSI-Prevenzione Investigators. Dietary supplementation with n-3 polyunsaturated fatty acids and vitamin E after myocardial infarction: results of the GISSI-Prevenzione trial. (1999) Lancet 354, 447-455.
25 Harris, W. S. Fish oils and plasma lipid and lipoprotein metabolism in humans: a critical review. (1989) J Lipid Res 30, 785-807.
26 Carey, M. C., Small, D. M. and Bliss, C. M. Lipid digestion and absorption. (1983) Annu Rev Physiol 45, 651-677.

1. A nutritional supplement comprising a sterol and an omega-3 fatty acid, or an ester thereof, for lowering cholesterol and triglyceride levels in the bloodstream of a subject.

2. The nutritional supplement according to claim 1, wherein the sterol and omega-3 fatty acid are together in the form of an ester.

3. The nutritional supplement according to claim 1, wherein the omega-3 fatty acid, that is present as such or as a component of an ester, has the formula: 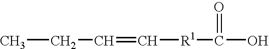
wherein R¹ is a (C₃-C₄₀) alkenyl group comprising at least one double bond.

4. The nutritional supplement according to claim 3, wherein R¹ has from 2 to 5 double bonds.

5. The nutritional supplement according to claim 2, wherein the omega-3 fatty acid is eicosapentaenoic acid 20:5ω3 (EPA).

6. The nutritional supplement according to claim 2, wherein the omega-3 fatty acid is docosahexaenoic acid 22:6ω3 (DHA).

7. The nutritional supplement according to claim 2, wherein the sterol is stigmasterol.

8. The nutritional supplement according to claim 2, wherein the sterol is sitosterol.

9. The nutritional supplement according to claim 2, wherein the sterol is fucosterol.

10. The nutritional supplement according to claim 2, wherein the sterol is fucostanol.

11. The nutritional supplement according to claim 2, wherein the sterol is β-sitostanol.

12. The nutritional supplement according to claim 1, wherein the sterol is a phytosterol.

13. The nutritional supplement according to claim 1, wherein the omega-3 fatty acid is derived from fish oil.

14. A method of lowering cholesterol and triglyceride levels in the bloodstream of a subject, the method including the step of administering an effective amount of a nutritional supplement comprising a sterol and an omega-3 fatty acid, or an ester thereof, to a subject.

15. The method according to claim 14, wherein the sterol and omega-3 fatty acid are together in the form of an ester.

16. The method according to claim 15, wherein the omega-3 fatty acid, that is present as such or as a component of an ester, has the formula: 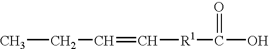
wherein R¹ is a (C₃-C₄₀) alkenyl group comprising at least one double bond.

17. The method according to claim 16, wherein R¹ has from 2 to 5 double bonds.

18. The method according to claim 15, wherein the omega-3 fatty acid is eicosapentaenoic acid 20:5ω3 (EPA).

19. The method according to claim 15, wherein the omega-3 fatty acid is docosahexaenoic acid 22:6ω3 (DHA).

20. The method according to claim 15, wherein the sterol is stigmasterol.

21. The method according to claim 15, wherein the sterol is sitosterol.

22. The method according to claim 15, wherein the sterol is fucosterol.

23. The method according to claim 15, wherein the sterol is fucostanol.

24. The method according to claim 15, wherein the sterol is β-sitostanol.

25. The method according to claim 15, wherein the sterol is a phytosterol.

26. The method according to claim 15, wherein the omega-3 fatty acid is derived from fish oil.

27. Use of a nutritional supplement comprising a sterol and an omega-3 fatty acid, or an ester thereof, for lowering cholesterol and triglyceride levels in the bloodstream of a subject.

28. A foodstuff having a nutritional value enhanced by incorporation of the nutritional supplement according to claim 2.

29. Use of the nutritional supplement according to claim 2 in the manufacture of a foodstuff.

30. A composition comprising a sterol ester of an omega-3 fatty acid, wherein the omega-3 fatty acid comprises eicosapentaenoic acid 20:5ω3 (EPA), docosahexaenoic acid 22:6ω3 (DHA) or stearidonic acid 18:4ω3 (SA), and the sterol comprises stigmasterol.

31. The composition of claim 30, wherein the omega-3 fatty acid is eicosapentaenoic acid 20:5ω3 (EPA).

32. The composition of claim 30, wherein the omega-3 fatty acid is docosahexaenoic acid 22:6ω3 (DHA).

33. The composition of claim 30, wherein the omega-3 fatty acid is stearidonic acid 18:4ω3 (SA).

34. The composition of claim 30, wherein the omega-3 fatty acid comprises a mixture of eicosapentaenoic acid 20:5ω3 (EPA) and docosahexaenoic acid 22:6ω3 (DHA), and the sterol comprises stigmasterol.

35. A nutritional supplement comprising a sterol ester of an omega-3 fatty acid, wherein the omega-3 fatty acid comprises eicosapentaenoic acid 20:5ω3 (EPA), docosahexaenoic acid 22:6ω3 (DHA) or stearidonic acid 18:4ω3 (SA), and the sterol comprises stigmasterol.

36. The nutritional supplement of claim 35, wherein the omega-3 fatty acid is eicosapentaenoic acid 20:5ω3 (EPA).

37. The nutritional supplement of claim 35, wherein the omega-3 fatty acid is docosahexaenoic acid 22:6ω3 (DHA).

38. The nutritional supplement of claim 35, wherein the omega-3 fatty acid is stearidonic acid 18:4ω3 (SA).

39. The nutritional supplement of claim 35, wherein the omega-3 fatty acid comprises a mixture of eicosapentaenoic acid 20:5ω3 (EPA) and docosahexaenoic acid 22:6ω3 (DHA), and the sterol comprises stigmasterol.
